(19) United States
(12) Patent Application Publication
(10) Pub. No.: US 2025/0383317 A1
(43) Pub. Date: Dec. 18, 2025

(54) SENSOR ELEMENT AND GAS SENSOR

(71) Applicant: NGK INSULATORS, LTD., Nagoya-shi (JP)

(72) Inventors: Yuki ISHIHARA, Nagoya-shi (JP); Takashi HINO, Kasugai-shi (JP)

(21) Appl. No.: 19/234,333

(22) Filed: Jun. 11, 2025

(30) Foreign Application Priority Data
Jun. 13, 2024 (JP) 2024-095786

(51) Int. Cl. G01N 27/407; G01N 27/41; G01N 33/00

(52) U.S. Cl. CPC G01N 27/4072; G01N 27/41; G01N 33/0027

(57) ABSTRACT

The sensor element includes a diffusion control section that provides the measurement-object gas introduced from the gas inlet with diffusion resistance, wherein the diffusion control section includes a space portion through which the measurement-object gas is caused to flow in the first direction, a shape of a cross-section perpendicular to the first direction in at least a part of the space portion in the first direction is a predetermined shape with a longer outer circumference than a reference rectangle, and the reference rectangle is a rectangle that has the same area as the cross-section of the space portion, and the reference rectangle is a rectangle that is formed by deforming the circumscribed rectangle of the cross-section, which is composed of two sides parallel to the layered direction and two sides parallel to a second direction that is perpendicular to the layered direction.

### CROSS-REFERENCE TO RELATED APPLICATION

The present application claims priority from Japanese application JP2024-095786, filed on Jun. 13, 2024, the content of which is hereby incorporated by reference into this application.

### BACKGROUND OF THE INVENTION

### 1. Field of the Invention

The present invention relates to a sensor element and a gas sensor.

### 2. Description of the Related Art

Hitherto, a gas sensor that detects the concentration of a specific gas, such as NOx, in a measurement-object gas, such as the exhaust gas of an internal combustion engine, is known. For example, PTL 1 describes a gas sensor comprising a sensor element that comprise an element body, a main pump cell, an auxiliary pump cell, and a measurement pump cell. The element body includes an oxygen-ion-conductive solid electrolyte layer and has a measurement-object gas flow section inside that introduces the measurement-object gas and causes the measurement-object gas to flow. The main pump cell includes an inner main pump electrode provided in a first internal cavity of the measurement-object gas flow section and an outer main pump electrode provided on an outside of the element body that is exposed to the measurement-object gas. The auxiliary pump cell includes an inner auxiliary pump electrode provided in a second internal cavity located downstream of the first internal cavity in the measurement-object gas flow section and an outer auxiliary pump electrode provided on an outside of the element body that is exposed to the measurement-object gas. The measurement pump cell includes an inner measurement pump electrode provided in a measurement chamber located downstream of the second internal cavity in the measurement-object gas flow section and an outer measurement pump electrode provided on an outside of the element body that is exposed to the measurement-object gas. When using this sensor element to detect NOx concentrations, the oxygen concentration in the measurement-object gas is adjusted in the first internal cavity and the second internal cavity by the main pump cell and the auxiliary pump cell. Next, NOx in the measurement-object gas with the oxygen concentration adjusted is reduced in the measurement chamber. The NOx concentration in the measurement-object gas is then detected based on the pump current that flows when the measurement pump cell pumps out the oxygen produced by the reduction of NOx.

### CITATION LIST

### Patent Literature

PTL 1: JP 2017-181499 A

### SUMMARY OF THE INVENTION

In such a gas sensor, even if a composition of the measurement-object gas is the same, an operation of a pump cell (e.g. a value of a pump current flowing through a pump cell of the sensor element) may differ if a dynamic pressure of the measurement-object gas differs. As a result, the dynamic pressure of the measurement-object gas may affect the accuracy in detection of the specific gas concentration. Therefore, it is desirable to reduce the effect of the dynamic pressure of measurement-object gas on the pump cell.

The present invention has been devised to solve such a problem, and it is a main object to reduce the effect of the dynamic pressure of measurement-object gas on the pump cell.

The present invention employs the following device to achieve the above-described main object.

[1] A sensor element according to the present invention is a sensor element for detecting a concentration of a specific gas in a measurement-object gas, the sensor element comprising: an element body having a longitudinal direction, being a layered body that is composed of multiple layers stacked in a layered direction perpendicular to the longitudinal direction, the multiple layers including an oxygen-ion-conductive solid electrolyte layer, and having a measurement-object gas flow section inside that introduces the measurement-object gas from a gas inlet and causes the measurement-object gas to flow in a first direction; a pump cell including an inner electrode provided in an internal cavity of the measurement-object gas flow section and an outer electrode provided on an outer surface of the element body, and a diffusion control section that is located upstream of the internal cavity in the measurement-object gas flow section and that provides the measurement-object gas introduced from the gas inlet with diffusion resistance, wherein the diffusion control section includes a space portion through which the measurement-object gas is caused to flow in the first direction, a shape of a cross-section perpendicular to the first direction in at least a part of the space portion in the first direction is a predetermined shape with a longer outer circumference than a reference rectangle, and the reference rectangle is a rectangle that has the same area as the cross-section of the space portion, and the reference rectangle is a rectangle that is formed by deforming the circumscribed rectangle of the cross-section, which is composed of two sides parallel to the layered direction and two sides parallel to a second direction that is perpendicular to the layered direction, so that short sides become shorter while maintaining the length of long sides, or a rectangle that is formed by deforming the circumscribed rectangle while maintaining a ratio of each side, or a rectangle that is formed by changing the length of each side of the circumscribed rectangle so as to maximize the overlapping area with the cross-section.

In this sensor element, the diffusion control section includes the space portion through which the measurement-object gas is caused to flow in the first direction. In addition, the shape of the cross-section perpendicular to the first direction in at least a part of the space portion in the first direction is a predetermined shape with a longer outer circumference than a reference rectangle. As a result, a hydraulic diameter of the part of the space portion of the diffusion control section that has the predetermined shape becomes smaller than a hydraulic diameter of the reference rectangle, and the pressure loss of the measurement-object gas passing through the space portion becomes higher. Therefore, the effect of the dynamic pressure of measurement-object gas on the pump cell including the inner electrode that is located downstream of a diffusion control section can be reduced.

[2] In the above-described sensor element (the sensor element according to [1] above), the outer circumference of the predetermined shape may include a first portion and a second portion that face each other in the layered direction and extend along the second direction, respectively, and at least a part of the first portion and at least a part of the second portion may each have a curved shape.

[3] In the above-described sensor element (the sensor element according to [1] or [2] above), the outer circumference of the predetermined shape may include a first portion and a second portion that face each other in the layered direction and extend along the second direction, respectively, and at least a part of the first portion and/or at least a part of the second portion may each have a polygonal line shape.

[4] In the above-described sensor element (the sensor element according to [2] or [3] above), the first portion and the second portion may each have a convex shape on the same side in the layered direction.

[5] In the above-described sensor element (the sensor element according to [2] or [3] above), the first portion and the second portion may each have a convex shape toward a side where they approach each other or toward a side where they are separated from each other in the layered direction.

[6] In the above-described sensor element (the sensor element according to any one of [1] to [5] above), the cross-section of the space portion may have a length in the second direction that is greater than a length in the layered direction.

[7] In the above-described sensor element (the sensor element according to any one of [1] to [6] above), the diffusion control section may have a plurality of the space portions, and each of the plurality of the space sections may have a shape of the cross-section that is the predetermined shape.

[8] In the above-described sensor element (the sensor element according to any one of [1] to [7] above), the space portion may be formed such that a cross-sectional area of a cross-section perpendicular to the first direction is smaller in at least a part of a downstream side of upstream end than in the upstream end. This increases the pressure loss of the measurement-object gas passing through a portion of the space portion having a smaller cross-sectional area than the upstream end. Therefore, by this shape also, the effect of the dynamic pressure of measurement-object gas on the pump cell including the inner electrode that is located downstream of a diffusion control section can be reduced.

[9] In the above-described sensor element (the sensor element according to [8] above), the space portion may have an outline of cross-section parallel to the first direction, and the outline may extend so as to curve with respect to the first direction.

[10] A gas sensor according to the present invention comprises the sensor element according to any one of [1] to [9]. Thus, the gas sensor produces the same advantageous effects as those produced by the above sensor element. For example, an advantageous effect is the reduction of the effect of the dynamic pressure of measurement-object gas on the pump cell.

### BRIEF DESCRIPTION OF THE DRAWINGS

FIG. 1 is a cross-sectional schematic view schematically showing an example of a configuration of a gas sensor 100.

FIG. 2 is a view along the arrow A in FIG. 1.

FIG. 3 is a cross-sectional view taken along the line B-B in FIG. 1.

FIG. 4 is a block diagram showing an electrical connection relationship between a control apparatus 95 and relevant elements including cells.

FIG. 5 is a cross-sectional view taken along the line C-C in FIG. 3.

FIG. 6 is an explanatory diagram showing a reference rectangle S1.

FIGS. 7A to 7C are partial cross-sectional views showing a manufacturing process of a first space portion 85a of a first diffusion control section 11.

FIG. 8 is an explanatory diagram showing a reference rectangle S2.

FIG. 9 is an explanatory diagram showing a first space portion 85a according to a modification.

FIG. 10 is an explanatory diagram showing a first space portion 85a according to a modification.

FIG. 11 is an explanatory diagram showing a first space portion 85a according to a modification.

FIG. 12 is an explanatory diagram showing a first space portion 85a according to a modification.

FIG. 13 is an explanatory diagram showing a first space portion 85a according to a modification.

FIG. 14 is an explanatory diagram showing a first space portion 85a according to a modification.

FIG. 15 is an explanatory diagram showing a first space portion 85a according to a modification.

FIG. 16 is an explanatory diagram showing a first space portion 85a according to a modification.

FIG. 17 is an explanatory diagram showing a first space portion 85a according to a modification.

FIG. 18 is an explanatory diagram showing a first space portion 85a according to a modification.

FIG. 19 is a cross-sectional schematic view showing a first diffusion control section 211 according to a modification.

FIG. 20 is a cross-sectional view taken along the line D-D in FIG. 19.

FIG. 21 is a cross-sectional view taken along the line E-E in FIG. 20.

### DETAILED DESCRIPTION OF THE INVENTION

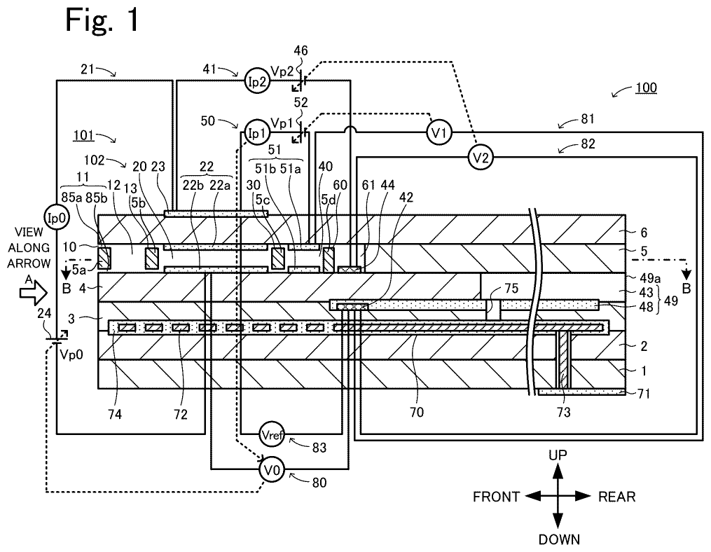

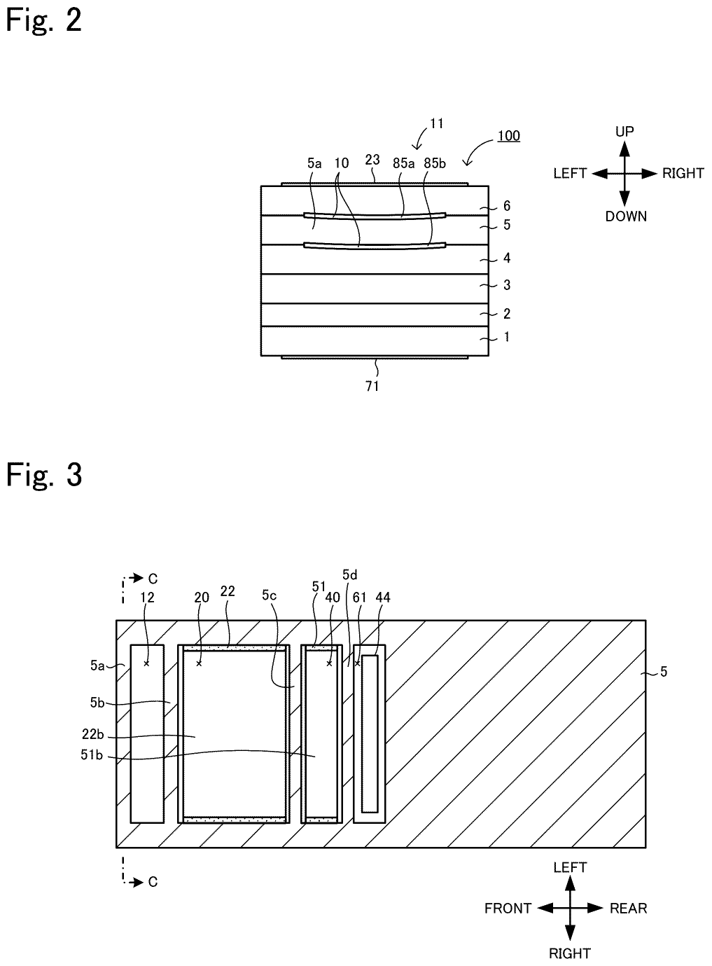

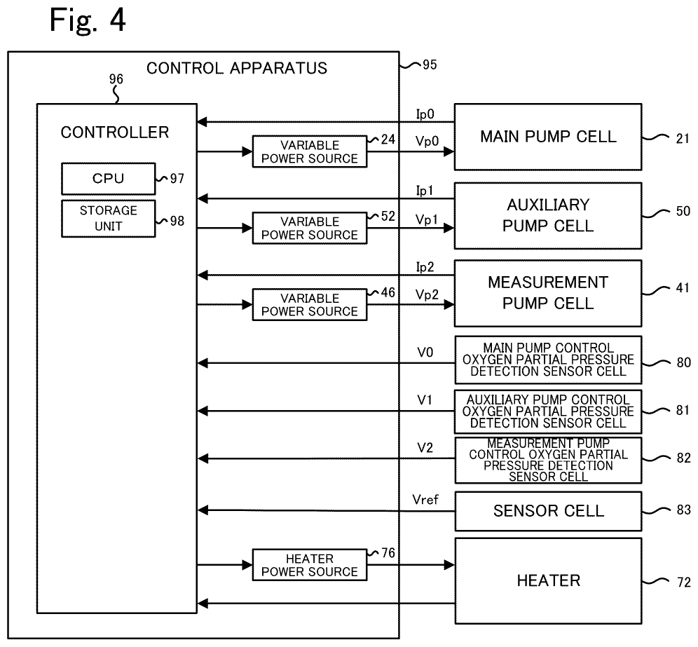

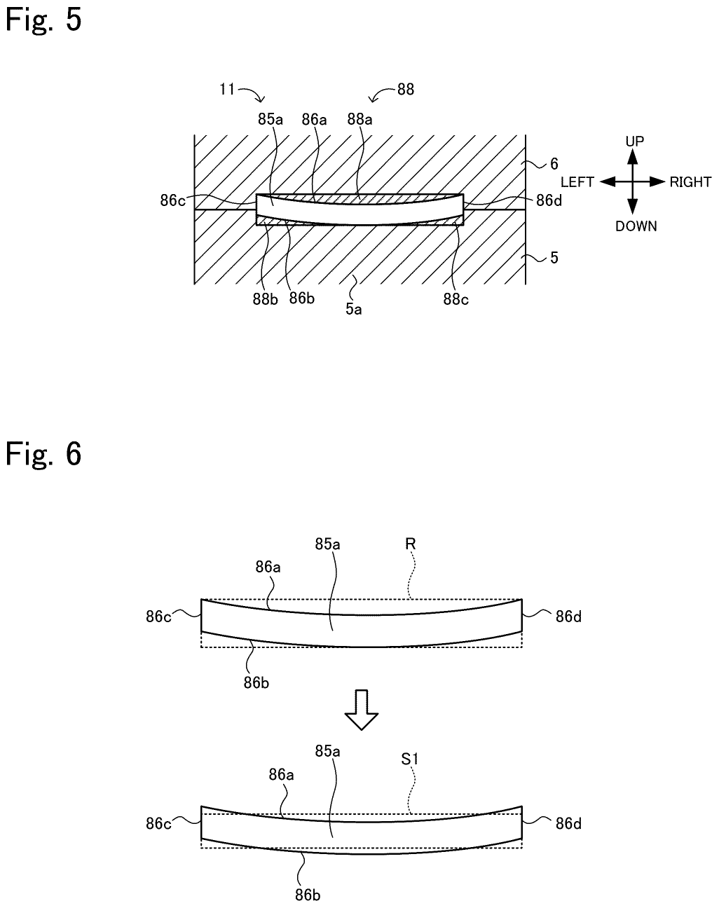

Next, an embodiment of the present invention will be described using the drawings. FIG. 1 is a cross-sectional schematic view schematically showing an example of the configuration of gas sensor 100 which is an embodiment of the present invention. FIG. 2 is a view along the arrow A in FIG. 1. FIG. 3 is a cross-sectional view taken along the line B-B in FIG. 1. FIG. 4 is a block diagram showing an electrical connection relationship between a control apparatus 95, cells and a heater 72. FIG. 5 is a cross-sectional view taken along the line C-C in FIG. 3. FIG. 5 is a cross-sectional schematic view schematically showing an area around a first space portion 85a of a first diffusion control section 11. The gas sensor 100 is installed in a pipe, such as an exhaust gas pipe of an internal combustion engine, for example. The gas sensor 100 uses the exhaust gas from an internal combustion engine as the measurement-object gas, and detects specific gas concentration which is the concentration of a specific gas such as NOx or ammonia in the measurement-object gas. In the present embodiment, the gas sensor 100 measures a NOx concentration as the specific gas concentration. The gas sensor 100 has a sensor element 101 including a long rectangular parallelepiped element body 102, cells 21, 41, 50, 80 to 83 included in the sensor element 101, a heater portion 70 provided inside the sensor element 101, and a control apparatus 95 that includes variable power supplies 24, 46, 52 and a heater power source 76, and controls the entire gas sensor 100. In the present embodiment, as shown in FIGS. 1 to 3, the longitudinal direction of the element body 102 of the sensor element 101 is defined as the front-rear direction (length direction), the layered direction (thickness direction) of each layer 1 to 6 of the element body 102 is defined as the up-down direction, and the direction perpendicular to the front-rear direction and the up-down direction is defined as the left-right direction (width direction). FIG. 5 is illustrated with the up-down length stretched out for the sake of illustration, compared to FIG. 2.

The element body 102 is a layered body in which six layers, that is, a first substrate layer 1, a second substrate layer 2, a third substrate layer 3, a first solid electrolyte layer 4, a spacer layer 5, and a second solid electrolyte layer 6, each made up of an oxygen-ion-conductive solid electrolyte layer made of zirconia (ZrO₂) or the like, are laminated in this order from a lower side in the drawing. The solid electrolyte forming these six layers is a dense, airtight one. The element body 102 is manufactured by, for example, applying predetermined processing, printing of a circuit pattern, and the like on a ceramic green sheet corresponding to each layer, then laminating those sheets, and further firing the sheets to be integrated.

At a tip end portion side of the sensor element 101 (the element body 102) (left end portion side in FIG. 1), a gas inlet port 10, a first diffusion control section 11, a buffer space 12, a second diffusion control section 13, a first internal cavity (an oxygen concentration adjustment chamber) 20, a third diffusion control section 30, a second internal cavity (then oxygen concentration adjustment chamber) 40, a fourth diffusion control section 60, and a third internal cavity (a measurement chamber) 61, are formed adjacent to each other so as to communicate with each other in this order between the lower surface of the second solid electrolyte layer 6 and the upper surface of the first solid electrolyte layer 4.

The gas inlet port 10, the buffer space 12, the first internal cavity 20, the second internal cavity 40, and the third internal cavity 61 are spaces of which top parts, bottom parts, and side parts, provided by hollowing the spacer layer 5, are respectively defined by the lower surface of the second solid electrolyte layer 6, the upper surface of the first solid electrolyte layer 4, and the side surface of the spacer layer 5 inside the sensor element 101.

Each of the first diffusion control section 11, the second diffusion control section 13, and the third diffusion control section 30 is provided as two laterally long slits (openings of which the longitudinal direction is a direction perpendicular to the drawing). FIGS. 2 and 5 illustrate the shapes of the first space portion 85a and second space portion 85b which constitute the two slits of the first diffusion control section 11. The fourth diffusion control section 60 is provided as a single laterally long slit (an opening of which the longitudinal direction is a direction perpendicular to the drawing) formed as a clearance from the lower surface of the second solid electrolyte layer 6. A part from the gas inlet port 10 to the third internal cavity 61 is also referred to as measurement-object gas flow section.

The sensor element 101 (element body 102) includes a reference-gas introduction portion 49 that allows the reference gas to flow from outside the sensor element 101 to a reference electrode 42 in the measurement of NOx concentration. The reference-gas introduction portion 49 has a reference-gas introduction space 43 and a reference-gas introduction layer 48. The reference gas introduction space 43 is a space that is provided inward from a rear end face of the sensor element 101. The reference-gas introduction space 43 is provided at a position between the upper surface of the third substrate layer 3 and the lower surface of the spacer layer 5 and has lateral sides defined by the side surfaces of the first solid electrolyte layer 4. The reference-gas introduction space 43 has an opening at the rear end face of the sensor element 101. This opening functions as an entrance 49a of the reference-gas introduction portion 49. The reference gas is introduced into the reference-gas introduction space 43 through the entrance 49a. The reference-gas introduction portion 49 introduces the reference gas to the reference electrode 42 while applying a predetermined diffusion resistance to the reference gas received through the entrance 49a. In the present embodiment, the reference gas is ambient air.

The reference-gas introduction layer 48 is disposed between the upper surface of the third substrate layer 3 and the lower surface of the first solid electrolyte layer 4. The reference-gas introduction layer 48 is a porous body composed of a ceramic material such as alumina. A part of the upper surface of the reference-gas introduction layer 48 is exposed in the reference-gas introduction space 43. The reference-gas introduction layer 48 is provided over the reference electrode 42. The reference-gas introduction layer 48 allows the reference gas to flow from the reference-gas introduction space 43 to the reference electrode 42.

The reference electrode 42 is an electrode formed in such a manner in which the reference electrode 42 is sandwiched by the upper surface of the third substrate layer 3 and the first solid electrolyte layer 4. As described above, the reference gas inlet layer 48 that communicates with the reference gas inlet space 43 is provided around the reference electrode 42. As will be described later, it is possible to measure an oxygen concentration (oxygen partial pressure) in the first internal cavity 20, an oxygen concentration (oxygen partial pressure) in the second internal cavity 40, and an oxygen concentration (oxygen partial pressure) in the third internal cavity 61 by using the reference electrode 42. The reference electrode 42 is formed as a porous cermet electrode (for example, a cermet electrode of Pt and ZrO₂).

In the measurement-object gas flow section, the gas inlet port 10 is a portion that is open to an external space, and a measurement-object gas is taken into the sensor element 101 from the external space through the gas inlet port 10. The first diffusion control section 11 is a portion that applies predetermined diffusion resistance to a measurement-object gas taken in through the gas inlet port 10. In the present embodiment, a front end of the first diffusion control section 11 is open to the front end surface of the sensor element 101, and this opening is the gas inlet 10. As illustrated in FIGS. 1 and 2, the first space portion 85a of the first diffusion control section 11 is provided between an upper surface of a first bridging portion 5a of the spacer layer 5 and the bottom surface of the second solid electrolyte layer 6. The second space portion 85b is provided between a bottom surface of the first bridging portion 5a and the upper surface of the first solid electrolyte layer 4. As illustrated in FIG. 3, the first bridging portion 5a is a portion that bridges a front side of the buffer space 12, which is a portion of the spacer layer 5 that has been punched out, in the left-right direction. The buffer space 12 is a space provided to guide the measurement-object gas introduced from the first diffusion control section 11 to the second diffusion control section 13. The second diffusion control section 13 is a portion that applies predetermined diffusion resistance to the measurement-object gas introduced from the buffer space 12 into the first internal cavity 20. As illustrated in FIGS. 1 and 2, the second diffusion control section 13 is provided between an upper surface of a second bridging portion 5b of the spacer layer 5 and the lower surface of the second solid electrolyte layer 6, and between a lower surface of the second bridging portion 5b and the upper surface of the first solid electrolyte layer 4. As illustrated in FIG. 3, the second bridging portion 5b is a portion that bridges between the buffer space 12 and the first internal cavity 20, which are portions of the spacer layer 5 that have been punched out respectively, in the left-right direction. When the measurement-object gas is introduced from the outside of the sensor element 101 into the first internal cavity 20, the measurement-object gas rapidly taken into the sensor element 101 through the gas inlet port 10 due to pressure fluctuations of the measurement-object gas in the external space (due to pulsation of exhaust pressure when the measurement-object gas is the exhaust gas of an automobile) is not directly introduced into the first internal cavity 20 but, after pressure fluctuations of the measurement-object gas are cancelled out through the first diffusion control section 11, the buffer space 12, and the second diffusion control section 13, the measurement-object gas is introduced into the first internal cavity 20. With this configuration, pressure fluctuations of the measurement-object gas introduced into the first internal cavity 20 are almost ignorable. The first internal cavity 20 is provided as a space used to adjust an oxygen partial pressure in the measurement-object gas introduced through the second diffusion control section 13. The oxygen partial pressure is adjusted by the operation of a main pump cell 21.

The main pump cell 21 is an electrochemical pump cell constituted of an inner pump electrode 22 including a ceiling electrode portion 22a disposed on the lower surface of the second solid electrolyte layer 6 over substantially the entirety of an area that faces the first internal cavity 20; an outer pump electrode 23 disposed on the upper surface of the second solid electrolyte layer 6 over an area that corresponds to the ceiling electrode portion 22a in such a manner as to be exposed to the outside of the sensor element 101; and the second solid electrolyte layer 6, the spacer layer 5, and the first solid electrolyte layer 4 that form a current path between the electrodes 22 and 23.

The inner pump electrode 22 is formed over the upper and lower solid electrolyte layers (the second solid electrolyte layer 6 and the first solid electrolyte layer 4) defining the first internal cavity 20, and the spacer layer 5 providing a side wall. Specifically, the ceiling electrode portion 22a is formed on the lower surface of the second solid electrolyte layer 6, providing a ceiling surface of the first internal cavity 20, a bottom electrode portion 22b is formed on the upper surface of the first solid electrolyte layer 4, providing a bottom surface, a side electrode portion (not shown) is formed on the side wall surface (inner surface) of the spacer layer 5, making both side wall portions of the first internal cavity 20, so as to connect those ceiling electrode portion 22a and the bottom electrode portion 22b, and the inner pump electrode 22 is disposed with a structure in a tunnel form at a portion where the side electrode portion is disposed.

The inner pump electrode 22 and the outer pump electrode 23 each are formed as a porous cermet electrode (for example, a cermet electrode of Pt and ZrO₂, having an Au content of 1 percent). The inner pump electrode 22 that contacts with a measurement-object gas is formed by using a material of which the reduction ability for NOx components in the measurement-object gas is lowered.

By passing a pump current Ip0 in a positive direction or a negative direction between the inner pump electrode 22 and the outer pump electrode 23 by applying a desired voltage Vp0 between the inner pump electrode 22 and the outer pump electrode 23, the main pump cell 21 is capable of pumping out oxygen in the first internal cavity 20 to the external space or pumping oxygen in the external space into the first internal cavity 20.

In order to detect an oxygen concentration (oxygen partial pressure) in an atmosphere in the first internal cavity 20, an electrochemical sensor cell, that is, a main pump control oxygen partial pressure detection sensor cell 80, is made up of the inner pump electrode 22, the second solid electrolyte layer 6, the spacer layer 5, the first solid electrolyte layer 4, the third substrate layer 3, and the reference electrode 42.

An oxygen concentration (oxygen partial pressure) in the first internal cavity 20 is found by measuring an electromotive force (voltage V0) in the main pump control oxygen partial pressure detection sensor cell 80. In addition, the pump current Ip0 is controlled by executing feedback control over the voltage Vp0 of a variable power source 24 such that the voltage V0 becomes a target value. With this configuration, it is possible to maintain the oxygen concentration in the first internal cavity 20 at a predetermined constant value.

The third diffusion control section 30 is a portion that applies predetermined diffusion resistance to a measurement-object gas of which the oxygen concentration (oxygen partial pressure) is controlled by operation of the main pump cell 21 in the first internal cavity 20 to guide the measurement-object gas to the second internal cavity 40. As illustrated in FIG. 1, the third diffusion control section 30 is provided between an upper surface of a third bridging portion 5c of the spacer layer 5 and the lower surface of the second solid electrolyte layer 6, and between a lower surface of the third bridging portion 5c and the upper surface of the first solid electrolyte layer 4. As illustrated in FIG. 3, the third bridging portion 5c is a portion that bridges between the first internal cavity 20 and the second internal cavity 40, which are portions of the spacer layer 5 that have been punched out respectively, in the left-right direction.

The second internal cavity 40 is provided as a space used to further adjust the oxygen partial pressure by using an auxiliary pump cell 50 for the measurement-object gas adjusted in the oxygen concentration (oxygen partial pressure) in the first internal cavity 20 in advance and then introduced through the third diffusion control section 30. With this configuration, it is possible to highly accurately maintain the oxygen concentration in the second internal cavity 40 at a constant value, so it is possible to measure a highly accurate NOx concentration with the gas sensor 100.

The auxiliary pump cell 50 is an auxiliary electrochemical pump cell made up of an auxiliary pump electrode 51 having a ceiling electrode portion 51a provided substantially all over the lower surface of the second solid electrolyte layer 6, facing the second internal cavity 40, the outer pump electrode 23 (not limited to the outer pump electrode 23, and an adequate electrode outside the sensor element 101 may be used), the second solid electrolyte layer 6, the spacer layer 5, and the first solid electrolyte layer 4.

The auxiliary pump electrode 51 is disposed in the second internal cavity 40 with a structure in a similar tunnel form to that of the inner pump electrode 22 provided in the above-described first internal cavity 20. In other words, the auxiliary pump electrode 51 has such a structure in a tunnel form that a ceiling electrode portion 51a is formed on the lower surface of the second solid electrolyte layer 6 providing the ceiling surface of the second internal cavity 40, a bottom electrode portion 51b is formed on the upper surface of the first solid electrolyte layer 4 providing the bottom surface of the second internal cavity 40, a side electrode portion (not shown) that couples those ceiling electrode portion 51a and bottom electrode portion 51b is formed on each of both wall surfaces of the spacer layer 5, providing a side wall of the second internal cavity 40. The auxiliary pump electrode 51, as well as the inner pump electrode 22, is formed by using a material of which the reduction ability for NOx components in the measurement-object gas is lowered.

By applying a desired voltage Vp1 between the auxiliary pump electrode 51 and the outer pump electrode 23, the auxiliary pump cell 50 is capable of pumping out oxygen in an atmosphere in the second internal cavity 40 to the external space or pumping oxygen from the external space into the second internal cavity 40.

In order to control an oxygen partial pressure in an atmosphere in the second internal cavity 40, an electrochemical sensor cell, that is, an auxiliary pump control oxygen partial pressure detection sensor cell 81, is made up of the auxiliary pump electrode 51, the reference electrode 42, the second solid electrolyte layer 6, the spacer layer 5, the first solid electrolyte layer 4, and the third substrate layer 3.

The auxiliary pump cell 50 performs pumping with a variable power source 52 of which the voltage is controlled in accordance with an electromotive force (voltage V1) detected by the auxiliary pump control oxygen partial pressure detection sensor cell 81. With this configuration, the oxygen partial pressure in an atmosphere in the second internal cavity 40 is controlled to a low partial pressure that substantially does not influence measurement of NOx.

Together with this, its pump current Ip1 is used to control the electromotive force of the main pump control oxygen partial pressure detection sensor cell 80. Specifically, the pump current Ip1 is input to the main pump control oxygen partial pressure detection sensor cell 80 as a control signal, and the gradient of the oxygen partial pressure in the measurement-object gas to be introduced from the third diffusion control section 30 into the second internal cavity 40 is controlled to be constantly unchanged by controlling the above-described target value of the voltage V0. When used as a NOx sensor, the oxygen concentration in the second internal cavity 40 is maintained at a constant value of about 0.001 ppm by the functions of the main pump cell 21 and auxiliary pump cell 50.

The fourth diffusion control section 60 is a portion that applies predetermined diffusion resistance to the measurement-object gas of which the oxygen concentration (oxygen partial pressure) is controlled by operation of the auxiliary pump cell 50 in the second internal cavity 40 to guide the measurement-object gas to the third internal cavity 61. The fourth diffusion control section 60 plays a role in limiting the amount of NOx flowing into the third internal cavity 61. As illustrated in FIG. 1, the fourth diffusion control section 60 is provided between an upper surface of a fourth bridging portion 5d of the spacer layer 5 and the lower surface of the second solid electrolyte layer 6, and between a lower surface of the fourth bridging portion 5d and the upper surface of the first solid electrolyte layer 4. As illustrated in FIG. 3, the fourth bridging portion 5d is a portion that bridges between the second internal cavity 40 and the third internal cavity 61, which are portions of the spacer layer 5 that have been punched out respectively, in the left-right direction.

The third internal cavity 61 is provided as a space used to perform a process related to measurement of a nitrogen oxide (NOx) concentration in a measurement-object gas on the measurement-object gas adjusted in oxygen concentration (oxygen partial pressure) in the second internal cavity 40 in advance and then introduced through the fourth diffusion control section 60. Measurement of a NOx concentration is mainly performed by operation of a measurement pump cell 41 in the third internal cavity 61.

The measurement pump cell 41 measures a NOx concentration in the measurement-object gas in the third internal cavity 61. The measurement pump cell 41 is an electrochemical pump cell made up of a measurement electrode 44 provided on the upper surface of the first solid electrolyte layer 4, facing the third internal cavity 61, the outer pump electrode 23, the second solid electrolyte layer 6, the spacer layer 5, and the first solid electrolyte layer 4. The measurement electrode 44 is a porous cermet electrode made of a material of which the reduction ability for NOx components in the measurement-object gas is raised as compared to the inner pump electrode 22. The measurement electrode 44 also functions as a NOx reduction catalyst that reduces NOx present in an atmosphere in the third internal cavity 61.

The measurement pump cell 41 is capable of pumping out oxygen produced as a result of decomposition of nitrogen oxides in an atmosphere around the measurement electrode 44 and detecting the amount of oxygen produced as a pump current Ip2.

In order to detect an oxygen partial pressure around the measurement electrode 44, an electrochemical sensor cell, that is, a measurement pump control oxygen partial pressure detection sensor cell 82, is made up of the first solid electrolyte layer 4, the third substrate layer 3, the measurement electrode 44, and the reference electrode 42. A variable power source 46 is controlled in accordance with an electromotive force (voltage V2) detected by the measurement pump control oxygen partial pressure detection sensor cell 82.

A measurement-object gas guided into the second internal cavity 40 reaches the measurement electrode 44 in the third internal cavity 61 through the fourth diffusion control section 60 in a situation in which the oxygen partial pressure is controlled. Nitrogen oxides in the measurement-object gas around the measurement electrode 44 are reduced (2NO→N₂+O₂) to produce oxygen. The produced oxygen is to be pumped by the measurement pump cell 41. At this time, the voltage Vp2 of the variable power source 46 is controlled such that the voltage V2 detected by the measurement pump control oxygen partial pressure detection sensor cell 82 is constant (target value). The amount of oxygen produced around the measurement electrode 44 is proportional to the concentration of nitrogen oxides in the measurement-object gas, so a nitrogen oxide concentration in the measurement-object gas is calculated by using the pump current Ip2 in the measurement pump cell 41.

When an oxygen partial pressure detection device is constructed as an electrochemical sensor cell by combining the measurement electrode 44, the first solid electrolyte layer 4, the third substrate layer 3, and the reference electrode 42, an electromotive force corresponding to the difference between the amount of oxygen produced by reduction of the NOx component in the atmosphere around the measurement electrode 44, and the amount of oxygen contained in the reference gas can be detected, and accordingly, the concentration of the NOx component in the measurement-object gas can be determined.

In addition, an electrochemical sensor cell 83 is made up of the second solid electrolyte layer 6, the spacer layer 5, the first solid electrolyte layer 4, the third substrate layer 3, the outer pump electrode 23, and the reference electrode 42, and it is possible to detect an oxygen partial pressure in a measurement-object gas outside the sensor by using an electromotive force (voltage Vref) obtained by the sensor cell 83.

In the gas sensor 100 having such a configuration, a measurement-object gas of which the oxygen partial pressure is maintained at a constantly unchanged low value (a value that substantially does not influence measurement of NOx) is supplied to the measurement pump cell 41 by operating the main pump cell 21 and the auxiliary pump cell 50. Therefore, it is possible to find a NOx concentration in the measurement-object gas in accordance with a pump current Ip2 that flows as a result of pumping out oxygen, produced by reduction of NOx, by the measurement pump cell 41 substantially in proportion to a NOx concentration in the measurement-object gas.

The sensor element 101 includes the heater portion 70 that plays a role in temperature adjustment for maintaining the temperature of the sensor element 101 by heating in order to increase the oxygen ion conductivity of the solid electrolyte. The heater portion 70 includes a heater connector electrode 71, a heater 72, a through-hole 73, a heater insulating layer 74, and a pressure release hole 75.

The heater connector electrode 71 is an electrode formed in such a manner as to be in contact with the lower surface of the first substrate layer 1. Connection of the heater connector electrode 71 to a heater power source 76 (see FIG. 2) allows electric power to be supplied from the heater power source 76 to the heater portion 70.

The heater 72 is an electric resistor formed in such a manner as to be sandwiched by the second substrate layer 2 and the third substrate layer 3 from upper and lower sides. The heater 72 is connected to the heater connector electrode 71 via the through-hole 73, and is supplied with electric power from a heater power source 76 to generate heat to increase and retain the temperature of the solid electrolyte forming the sensor element 101.

The heater 72 is embedded all over the region from the first internal cavity 20 to the third internal cavity 61, and is capable of adjusting the overall sensor element 101 to a temperature at which the solid electrolyte is activated.

The heater insulating layer 74 is an electrically insulating layer formed of an insulating material, such as alumina, on the top and lower surfaces of the heater 72. The heater insulating layer 74 is formed for the purpose of obtaining an electrical insulation property between the second substrate layer 2 and the heater 72 and an electrical insulation property between the third substrate layer 3 and the heater 72.

The pressure release hole 75 is a portion provided so as to extend through the third substrate layer 3 and the reference gas inlet layer 48 and communicate with the reference gas inlet space 43. The pressure release hole 75 is formed for the purpose of easing an increase in internal pressure resulting from an increase in temperature in the heater insulating layer 74.

As illustrated in FIG. 4, the control apparatus 95 includes the above-described variable power sources 24, 46, and 52, the above-described heater power source 76, and a controller 96. The controller 96 is a microprocessor including a CPU 97, a storage unit 98, and so forth. The storage unit 98 is an information-rewritable nonvolatile memory and is capable of storing, for example, various programs and various data. The controller 96 is configured to receive the voltage V0 of the main-pump-control oxygen-partial-pressure detection sensor cell 80, the voltage V1 of the auxiliary-pump-control oxygen-partial-pressure detection sensor cell 81, the voltage V2 of the measurement-pump-control oxygen-partial-pressure detection sensor cell 82, the voltage Vref of the sensor cell 83, the pump current Ip0 flowing through the main pump cell 21, the pump current Ip1 flowing through the auxiliary pump cell 50, and the pump current Ip2 flowing through the measurement pump cell 41. The controller 96 is configured to output control signals to the variable power sources 24, 46, and 52 and thus control the voltages Vp0, Vp1, and Vp2 that are to be output by the variable power sources 24, 46, and 52, thereby controlling the main pump cell 21, the measurement pump cell 41, and the auxiliary pump cell 50. The controller 96 is configured to output a control signal to the heater power source 76, thereby controlling the electric power to be supplied from the heater power source 76 to the heater 72. The storage unit 98 further stores target values V0*, V1*, V2*, and the like, to be described below. The CPU 97 of the controller 96 is configured to refer to the target values V0*, V1*, and V2* and thus control the pump cells 21, 41, and 50.

The controller 96 executes an auxiliary pump control process of controlling the auxiliary pump cell 50 so that the oxygen concentration in the second internal cavity 40 reaches a target concentration. Specifically, the controller 96 controls the auxiliary pump cell 50 by executing feedback control on the voltage Vp1 of the variable power source 52 so that the voltage V1 reaches a constant value (referred to as target value V1*). The target value V1* is defined as the value that causes the oxygen concentration in the second internal cavity 40 to reach a predetermined low oxygen concentration that does not substantially affect measurement of NOx.

The controller 96 executes a main pump control process of controlling the main pump cell 21 so that the pump current Ip1 flowing when the oxygen concentration in the second internal cavity 40 is adjusted by the auxiliary pump cell 50 in the auxiliary pump control process reaches a target current (referred to as target value Ip1*).

Specifically, the controller 96 sets (feedback-controls) a target value (referred to as a target value V0*) of the voltage V0 based on the pump current Ip1 so that the pump current Ip1 caused to flow by the voltage Vp1 reaches the constant target current Ip1*. The controller 96 then performs feedback control on the voltage Vp0 of the variable power source 24 so that the voltage V0 reaches the target value V0* (in other words, the oxygen concentration in the first internal cavity 20 reaches the target concentration). The gradient of oxygen partial pressure in the measurement-object gas to be introduced from the third diffusion control section 30 into the second internal cavity 40 is made unchanged constantly by the main pump control process. The target value V0* is set to a value which causes the oxygen concentration in the first internal cavity 20 to be higher than 0% and reach a low oxygen concentration. The pump current Ip0 which flows during the main pump control process varies according to the oxygen concentration in the measurement-object gas (that is, the measurement-object gas in the vicinity of the sensor element 101) which flows into the measurement-object gas flow section through the gas inlet port 10. Thus, the controller 96 can also detect the oxygen concentration in the measurement-object gas based on the pump current Ip0.

The main pump control process and the auxiliary pump control process described above are also collectively referred as an adjustment pump control process. The first internal cavity 20 and the second internal cavity 40 are also collectively referred as an oxygen concentration adjustment chamber. The main pump cell 21 and the auxiliary pump cell 50 are also collectively referred as an adjustment pump cell. The controller 96 executes the adjustment pump control process, thus the adjustment pump cell adjusts the oxygen concentration in the oxygen concentration adjustment chamber.

In addition, the controller 96 executes a measurement pump control process of controlling the measurement pump cell 41 so that the voltage V2 reaches a constant value (referred to as a target value V2*) (in other words, so that the oxygen concentration in the third internal cavity 61 reaches a predetermined low concentration). Specifically, the controller 96 controls the measurement pump cell 41 by performing feedback control on the voltage Vp2 of the variable power source 46 so that the voltage V2 reaches the target value V2*. Oxygen is pumped out from the third internal cavity 61 by the measurement pump control process.

Execution of the measurement pump control process causes oxygen to be pumped out from the third internal cavity 61 so that the oxygen produced due to reduction of NOx in the measurement-object gas in the third internal cavity 61 become substantially zero. The controller 96 obtains a pump current Ip2 as a detected value corresponding to the oxygen produced in the third internal cavity 61 from the specific gas (in this case, NOx), and calculates the NOx concentration in the measurement-object gas based on the pump current Ip2.

The storage unit 98 stores a relational expression (for example, an expression of a linear function or a quadratic function) or a map as a correspondence relationship between the pump current Ip2 and the NOx concentration. Such a relational expression or map can be determined in advance by an experiment.

The controller 96 performs a heater control process of controlling the heater 72 by outputting a control signal to the heater power source 76 so that the temperature of the heater 72 reaches a target temperature (for example, 800° C.). Here, the temperature of the heater 72 can be expressed as a linear function of the resistance value of the heater 72. Thus, in the heater control process, the controller 96 calculates the resistance value of the heater 72 as a value (a value convertible to the temperature) regarded as the temperature of the heater 72, and performs feedback control on the heater power source 76 so that the calculated resistance value reaches a target resistance value (a resistance value corresponding to the target temperature). The controller 96 obtains, for example, the voltage of the heater 72 and the current flowing through the heater 72, and can calculate the resistance value of the heater 72 based on the obtained voltage and current. The controller 96 may calculate the resistance value of the heater 72, for example, by 3-terminal method or 4-terminal method. When passing an electric current through the heater 72, the heater power source 76 adjusts the electric power supplied to the heater 72 by changing the value of the voltage to be applied to the heater 72 based on, for example, a control signal from the controller 96.

The control apparatus 95 inclusive of the variable power sources 24, 46, and 52, the heater power source 76, and so forth illustrated in FIG. 4 is actually connected to each electrode inside the sensor element 101 via lead wires not illustrated, which are formed inside the sensor element 101, and the connector electrodes not illustrated (only the heater connector electrode 71 is shown in FIG. 1), which are formed on the rear end side of the sensor element 101.

Here, the first diffusion control section 11 will be explained in detail. The first diffusion control section 11 is provided in the upstream side of the first internal cavity 20, the second internal cavity 40, and the third internal cavity 61 of the measurement-object gas flow section. As described above, the first diffusion control section 11 has the first space portion 85a and the second space portion 85b. The first space portion 85a and the second space portion 85b allow the measurement-object gas to flow in the front-rear direction (first direction). In the present embodiment, the second space portion 85b has the same shape as the first space portion 85a, so a detailed description of the second space portion 85b is omitted, and the shape of the first space portion 85a is described. As illustrated in FIG. 5, in a cross-section perpendicular to the front-rear direction, the first space portion 85a has the first to fourth portions 86a to 86d as a outer circumference of the cross-section. The first portion 86a, the second portion 86b, the third portion 86c, and the fourth portion 86d correspond to the upper, lower, left, and right sides of the outer circumference of the cross-section of the first space portion 85a. The first portion 86a and the second portion 86b face each other in the up-down direction (the layered direction) and extend along the left-right direction (the second direction), respectively. The third portion 86c and the fourth portion 86d face each other in the left-right direction and extend along the up-down direction, respectively. The first portion 86a and the second portion 86b each have a curved shape. The first portion 86a and the second portion 86b are each curved to form a convex shape on the same side in the up-down direction (in this case, lower side). The third portion 86c and the fourth portion 86d each have a straight line shape along the up-down direction. The cross-section of the first space portion 85a has a length in the left-right direction that is greater than a length in the up-down direction.

The element body 102 has a structural portion 88 that constitutes at least a part of the outer periphery of the first space portion 85a (an outer wall of the first diffusion control section 11). The structural portion 88 has a first structural portion 88a, a second structural portion 88b, and a third structural portion 88c. The first structural portion 88a is provided on the lower surface of the second solid electrolyte layer 6. The first structural portion 88a is thin at both left and right ends in the cross-section perpendicular to the front-rear direction and becomes thicker toward a center in the left-right direction. With this configuration, a lower surface of the first structural portion 88a thus curves so that the lower surface has a convex shape to the down direction, thereby constituting the first portion 86a of the first space portion 85a. The second structural portion 88b and the third structural portion 88c are provided on the upper surface of the spacer layer 5. The second structural portion 88b is thick at left end in the cross-section perpendicular to the front-rear direction and becomes thinner toward a center of the first space portion 85a in the left-right direction. The third structural portion 88c is thick at right end in the cross-section perpendicular to the front-rear direction and becomes thinner toward a center of the first space portion 85a in the left-right direction. With these configurations, an upper surface of the second structural portion 88b and an upper surface of the third structural portion 88c as a whole are curved so that the upper surfaces have a convex shape to the down direction, thereby constituting the second portion 86b. The third portion 86c and the fourth portion 86d that each have the straight line shape are constituted by not the structural portion 88 but the spacer layer 5 and second solid electrolyte layer 6, respectively. The structural portion 88 is made of a dense material that does not allow the measurement-object gas to flow through, and in the present embodiment is made of alumina ceramics. In FIGS. 1 and 2, the structural portion 88 is not illustrated. The first space portion 85a and the structural portion 88 each have the same shape (the shape shown in FIG. 5) in the cross-section perpendicular to the front-rear direction at any portion in the front-rear direction.

The cross-section of the first space portion 85a perpendicular to the front-rear direction has a predetermined shape whose outer circumference is longer than that of a reference rectangle. The reference rectangle will be described using FIG. 6. First, it is considered that a circumscribed rectangle R of the cross-section of the first space portion 85a, which is composed of two sides parallel to the up-down direction and two sides parallel to the left-right direction (upper part of FIG. 6). Next, the circumscribed rectangle R is modified by shortening the short sides while maintaining the length of the long sides so that the area is the same as that of the cross-section of the first space portion 85a, and the resulting rectangle is defined as a reference rectangle S1 (lower part of FIG. 6). The reference rectangle S1 has a shape corresponding to the conventional cross-sectional shape (rectangle) of the space of the first diffusion-controlling portion 11, and is a rectangle with the same cross-sectional area as the first space portion 85a so that it can be compared with the first space portion 85a. The cross-sectional area of the first space portion 85a is the same as the reference rectangle S1, and, unlike the reference rectangle S1, the first portion 86a and the second portion 86b each have the curved shape, so the length of the outer circumference of the first space portion 85a is longer than the reference rectangle S1. Furthermore, the greater the degree of curvature of the first portion 86a and second portion 86b, the longer the length of the outer circumference of the first space portion 85a can be made. Since the shape of the cross-section of the first space portion 85a is the predetermined shape with a longer outer circumference than a reference rectangle, a hydraulic diameter of the first space portion 85a is smaller than a hydraulic diameter of the reference rectangle S1. Here, the hydraulic diameter is expressed as four times the value obtained by dividing the cross-sectional area by the wetted perimeter (i.e., the length of the outer circumference of the cross-section) (the hydraulic diameter=cross-sectional area/wetted perimeter×4). Since the cross-sectional area of the first space portion 85a is equal to that of the reference rectangle S1, if the length of the outer circumference of the first space portion 85a (the sum of the lengths of the first to fourth portions 86a to 86d) is longer than the length of the outer circumference of the reference rectangle S1, the hydraulic diameter of the first space portion 85a will be smaller than that of the reference rectangle S1. Furthermore, the small hydraulic diameter increases the pressure loss of the measurement-object gas passing through the first space portion 85a. The effect of the dynamic pressure of the measurement-object gas on the main pump cell 21 including the inner pump electrode 22 that is located downstream of the diffusion control section 11 can be reduced. For example, even if a composition of the measurement-object gas is the same, an operation of the main pump cell 21 (e.g. a value of the pump current Ip0 flowing through the main pump cell 21 and so forth) may differ if the dynamic pressure of the measurement-object gas differs. As a result, the dynamic pressure of the measurement-object gas may affect the accuracy in detection of the specific gas concentration. In contrast, in the first diffusion control section 11 of the present embodiment, the cross-section of the first space portion 85a is formed into the predetermined shape to increase the pressure loss, thereby reducing the effect of the dynamic pressure of the measurement-object gas on the main pump cell 21. In addition, since not only the main pump cell 21, but also the auxiliary pump cell 50 and the measurement pump cell 41 also have auxiliary pump electrodes 51 and measurement electrodes 44 located downstream of the first diffusion control section 11, the effects on the auxiliary pump cell 50 and the measurement pump cell 41 of the dynamic pressure of the measurement-object gas can also be reduced by forming the cross-section of the first space portion 85a in the predetermined shape. The wording “rectangle” refers to a quadrilateral with all four angles being equal, and includes a square. If the circumscribed rectangle R is a square, it is sufficient to shorten either two sides of the two sides parallel to the up-down direction or the two sides parallel to the left-right direction within the circumscribing rectangle R.

In addition, it is also possible to increase the pressure loss by reducing the cross-sectional area. For example, even if the shape of the cross-section of the first space 85a is made smaller than the cross-sectional area while remaining the same rectangular shape as the reference rectangle S1, the pressure loss will be higher than that of the reference rectangle S1. However, if the cross-sectional area is reduced, the flow rate of the measurement-object gas passing through the first diffusion control section 11 itself will decrease, and for example, the pump currents Ip0, Ip1, and Ip2 flowing through the main pump cell 21, auxiliary pump cell 50, and measurement pump cell 41 downstream of the first diffusion control section 11 will always be in the range of small values, and the accuracy in detection of the specific gas concentration by the gas sensor 100 may decrease. In contrast, in the first diffusion control section 11 of the present embodiment, the cross-sectional area of the first space portion 85a is a predetermined shape, so it is possible to increase the pressure loss without reducing the cross-sectional area of the first space portion 85a compared to conventional shapes such as the reference rectangle S1. Therefore, it is possible to reduce the effects that the main pump cell 21 and so forth receive from the dynamic pressure of the measurement-object gas while maintaining the flow rate of the measurement-object gas passing through the first diffusion control section 11.

As mentioned above, the second space portion 85b has the same shape as the first space portion 85a, so that each of the first space portion 85a and the second space portion 85b of the first diffusion controlling section 11 has the shape of the cross-section perpendicular to the front-rear direction that is the predetermined shape.

Now, an example of method of manufacturing the sensor element 101 included in the gas sensor 100 will be described. First, six non-calcinated ceramic green sheets each containing an oxygen-ion-conductive solid electrolyte, as a ceramic component are prepared. In each of these green sheets, a plurality of sheet holes to be used for positioning during printing or stacking as well as necessary through-holes and the like are provided in advance. Furthermore, the green sheet that is to become the spacer layer 5 is preliminarily subjected to a punching process or the like in which a space that is to become the measurement-object gas flow section is provided. More specifically, by punching out the green sheet that is to become the spacer layer 5 in such a way as to leave the parts that is to become the first to fourth bridging portions 5a to 5d, spaces that is to become the buffer space 12, the first internal cavity 20, the second internal cavity 40, and the third internal cavity 61 are formed. Likewise, the green sheet that is to become the first solid electrolyte layer 4 is subjected to a process for providing a space that is to become the reference-gas introduction space 43. Then, a pattern-printing process and a drying process for forming various patterns in the ceramic green sheets are performed in correspondence with the first substrate layer 1, the second substrate layer 2, the third substrate layer 3, the first solid electrolyte layer 4, the spacer layer 5, and the second solid electrolyte layer 6. Specific patterns to be formed include, for example, patterns that serve as electrodes such as the measurement electrode 44 described above, the lead wires connected to the electrodes, the connector electrodes such as the heater connector electrode 71, the reference-gas introduction layer 48, and the heater section 70. The pattern-printing process is performed in which a pattern-forming paste prepared in accordance with the properties required for an object to be formed is applied onto a green sheet by using a known screen printing technique. The drying process is performed by using a known drying technique. When the pattern-printing process and the drying process are completed, a printing process and a drying process are performed in which a bonding paste for stacking and bonding together the green sheets corresponding to the respective layers are printed and dried. Subsequently, a stacking process is performed in which the green sheets provided with the bonding paste are stacked in a predetermined order while being positioned at the sheet holes. Next, a pressure bonding process is performed in which the stacked green sheets are put under predetermined temperature and pressure conditions to be pressure bonded into a single layered body. The layered body thus obtained contains a plurality of sensor elements 101 therein. The layered body is cut into pieces each having the size of the sensor element 101. Then, a calcinating process is performed in which the layered body is calcinated at a predetermined calcination temperature, whereby a sensor element 101 is obtained.

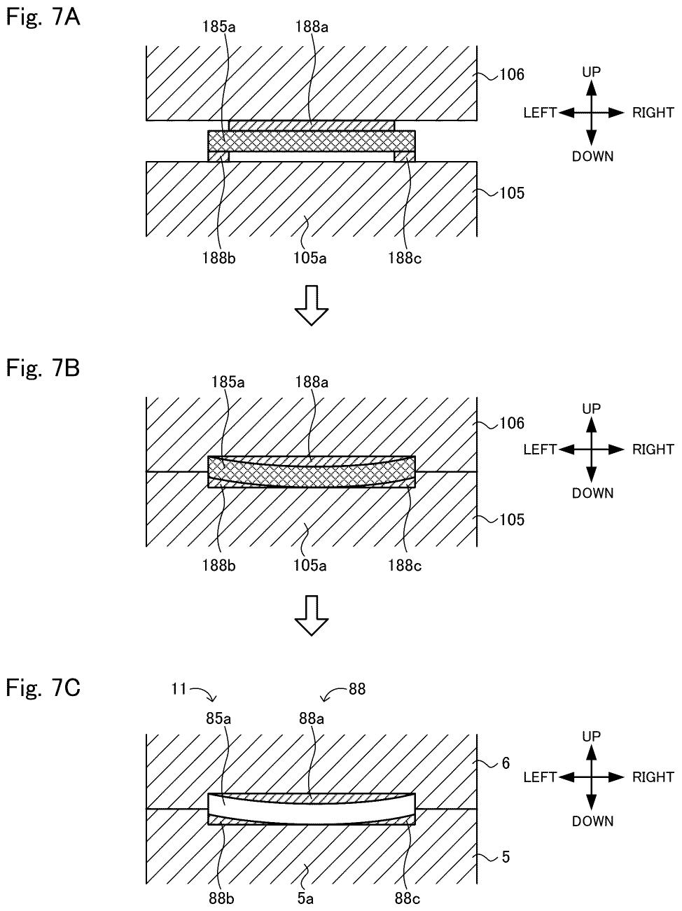

Here, a method of manufacturing the first diffusion control section 11 of the sensor element 101 will be described. FIGS. 7A to 7C are partial cross-sectional views showing a manufacturing process of a first space portion 85a of a first diffusion control section 11. Since the second space portion 85b can be manufactured using the same method of manufacturing as the first space portion 85a, a detailed description of a method of manufacturing the second space portion 85b is omitted, and a method of manufacturing the first space portion 85a is described. First, as illustrated in FIG. 7A, in the pattern-printing process described above, a second structural material layer 188b and a third structural material layer 188c, which is to become the second structural portion 88b and the third structural portion 88c respectively, are printed and formed on the upper surface of a bridging portion 105a (corresponding to the first bridging portion 5a) of a green sheet 105, which is to become the spacer layer 5. The second structural material layer 188b and the third structural material layer 188c are formed using a paste that contains alumina, for example as a ceramic component. The second structural material layer 188b and the third structural material layer 188c are formed that the second structural material layer 188b and the third structural material layer 188c tend to become thinner toward a center in the left-right direction as a whole, so that their upper surfaces will have a shape that corresponds to the curved shape of the second portion 86b of the first space portion 85a after the pressure bonding process. In the present embodiment, as illustrated in FIG. 7A, the second structural material layer 188b and the third structural material layer 188c are formed at the positions corresponding to the left and right ends of the first space portion 85a, respectively, on the upper surface of the first bridging portion 5a, and no structural material layer is formed in the area around the center of the first space portion 85a in the left-right direction. Next, a vanishing material layer 185a is printed and formed on the upper surface of the green sheet 105, in a region from a left edge of the second structural material layer 188b to a right edge of the third structural material layer 188c. The vanishing material layer 185a is a layer for forming the first space portion 85a, and is formed using a paste containing a material that burns away in the calcinating process described above (e.g. theobromine and so forth). Then, a first structural material layer 188a, which is to become the first structural portion 88a, is printed and formed on an upper surface of the vanishing material layer 185a. The first structural material layer 188a is formed using a paste that contains alumina as a ceramic component, in the same way as the second structural material layer 188b and the third structural material layer 188c. The first structural material layer 188a is formed so that the first structural material layer 188a tends to become thicker toward a center in the left-right direction, so that a lower surface of the first structural material layer 188a will have a shape that corresponds to the curved shape of the first portion 86a of the first space portion 85a after the pressure bonding process. In the present embodiment, as illustrated in FIG. 7A, a width of the first structural material layer 188a in the left-right direction is made smaller than a width of the vanishing material layer 185a in the left-right direction, and the first structural material layer 188a is not formed in positions corresponding to the left and right ends of the first space portion 85a. After printing is performed in this manner, the stacking process described above is performed, whereby a green sheet 106 which is to become the second solid electrolyte layer 6 is stacked on the first structural material layer 188a as illustrated in FIG. 7A.

The layered body is manufactured by performing the pressure bonding process described above from the state shown in FIG. 7A, resulting the state shown in FIG. 7B. In the layered body, as illustrated in FIG. 7B, since the green sheet 105 and the green sheet 106 are softer than the vanishing material layer 185a and the first to third structural material layers 188a to 188c, the upper surface of the green sheet 105 and the lower surface of the green sheet 106 are dented and the green sheet 105 and the green sheet 106 surround these layers from above, below, left and right. In addition, the vanishing material layer 185a and the first to third structural material layers 188a to 188c are deformed by being pressed from above and below by the green sheets 105 and 106, whereby the vanishing material layer 185a takes the shape of the first space portion 85a, and the first to third structural material layers 188a to 188c take the shapes of the first to third structural portions 88a to 88c. Viscosity (hardness) of the green sheets 105 and 106 can be adjusted, for example, by the kind or content ratio of a binder and a plasticizer contained in them. Viscosity (hardness) of the vanishing material layer 185a and the first to third structural material layers 188a to 188c can be adjusted, for example, by the solid content ratio or the content ratio of the binder in the paste used for printing these layers. By adjusting the viscosity (hardness) of them, the green sheets 105 and 106 can be dented as illustrated in FIG. 7B.

After that, the layered body is cut into the pieces each having the size of the sensor element 101, and the calcinating process described above is performed on the cut layered body, whereby the vanishing material layer 185a burns and disappears, and becomes the first space portion 85a, as illustrated in FIG. 7C. In addition, the green sheet 105, which includes the bridging portion 105a, and the green sheet 106 are calcinated and become the spacer layer 5, which includes the first bridging portion 5a, and the second solid electrolyte layer 6. The first to third structural material layers 188a to 188c are calcinated and become the first to third structural portions 88a to 88c. In this way, the first diffusion control section 11 including the first space portion 85a is manufactured.

The correspondence relationships between the components in the present embodiment and the components in the present invention will now be clarified. Layers 1 to 6 according to the present embodiment each correspond to the solid electrolyte layer according to the present invention. The element body 102 corresponds to the element body. The first internal cavity 20, the second internal cavity 40, and the third internal cavity 61 each correspond to the internal cavity. The inner pump electrode 22, the auxiliary pump electrode 51, and the measurement electrode 44 each correspond to the inner electrode. The outer pump electrode 23 corresponds to the outer electrode. The main pump cell 21, the auxiliary pump cell 50, and the measurement pump cell 41 each correspond to the pump cell. The first diffusion control section 11 corresponds to the diffusion control section. The first and second space portions 85a and 85b each correspond to the space portion. In addition, the first portion 86a corresponds to the first portion, and the second portion 86b corresponds to the second portion.

In the sensor element 101 included in the gas sensor 100 according to the present embodiment described in detail above, the diffusion control section 11 includes the first space portion 85a through which the measurement-object gas is caused to flow in the front-rear direction (the first direction). In addition, the shape of the cross-section perpendicular to the front-rear direction in the first space portion 85a is the predetermined shape with the longer outer circumference than the reference rectangle S1. With this configuration, the hydraulic diameter of the first space portion 85a becomes smaller than the hydraulic diameter of the reference rectangle S1, and the pressure loss of the measurement-object gas passing through the first space portion 85a becomes higher. Therefore, the effects of the dynamic pressure of measurement-object gas on the main pump cell 21, the auxiliary pump cell 50, and the measurement pump cell 41 that are located downstream of the first diffusion control section 11 can be reduced. Similarly, the shape of the cross-section perpendicular to the front-rear direction in the second space portion 85b is the predetermined shape with the longer outer circumference than the reference rectangle S1. Therefore, the pressure loss of the measurement-object gas passing through the second space portion 85b becomes higher, and the same effect as the first space portion 85a is obtained.

Note that the present invention is not limited to the above-described embodiment at all, and may be, of course, implemented in various modes within the technical scope of the present invention.

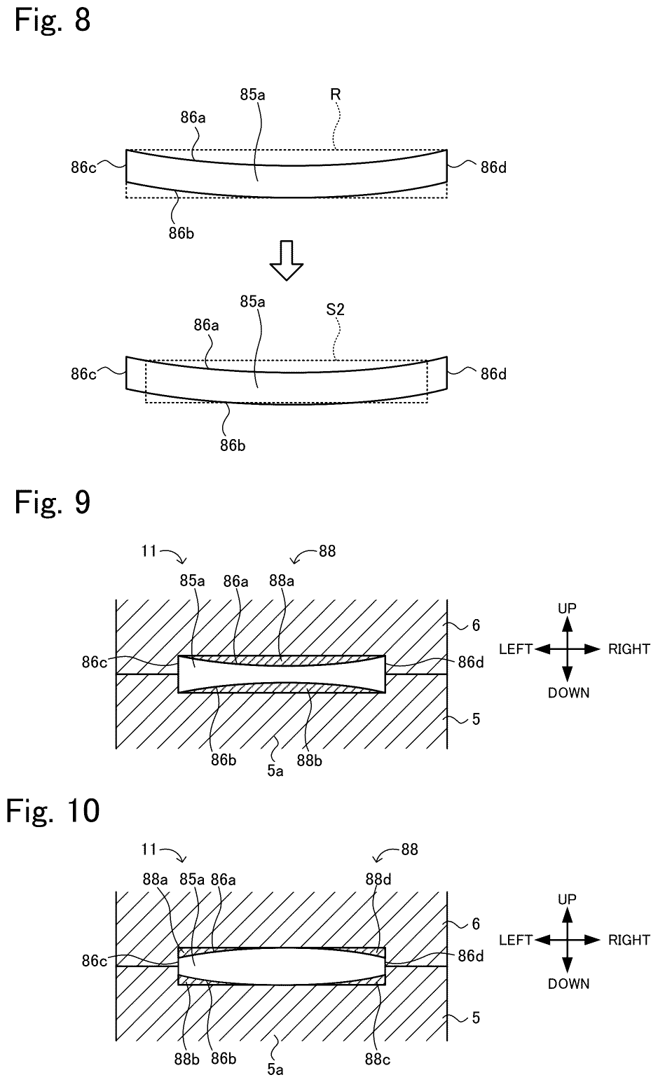

For example, in the embodiment described above, the reference rectangle S1 was defined as a rectangle with the same length as the long side and a shorter short side, so that the circumscribed rectangle R has the same cross-sectional area as the cross-sectional area of the first space portion 85a. However, the reference rectangle can be defined in other ways. For example, as illustrated in FIG. 8, the reference rectangle S2 (lower part of FIG. 8) may be a rectangle that is obtained so as to deform the circumscribed rectangle R of the first space portion 85a (upper part of FIG. 8) while maintaining the ratio of each side so that the cross-sectional area of the circumscribed rectangle R is the same as the cross-sectional area of the first space portion 85a. Alternatively, the circumscribing rectangle R may be a reference rectangle S3 (not shown in the figure) in which the length of each side has been changed so that the area is the same as the cross-sectional area of the first space portion 85a and the overlapping area with the cross-sectional area of the first space portion 85a is maximized. Note that the reference rectangle S3 may end up having the same shape as the reference rectangle S1 or the reference rectangle S2. If the shape of the cross-section perpendicular to the front-rear direction of the first space portion 85a is a predetermined shape with a longer outer circumference than at least one of the reference rectangles S1 to S3 defined in this way, the hydraulic diameter of the first space portion 85a will be smaller than that of the reference rectangle, and it can be said that the first space portion 85a has a shape that causes a higher pressure loss for the measurement-object gas passing through it. In the embodiment described above, the degree of curvature of the first portion 86a and second portion 86b is determined so that the cross-sectional shape of the first space portion 85a has a predetermined shape with a longer outer circumference than any of the reference rectangles S1 to S3.

In the embodiment described above, the first portion 86a and the second portion 86b of the first space portion 85a each have the curved shape, but it is sufficient if the cross-sectional shape of the first space portion 85a is the predetermined shape described above (the shape with a longer outer circumference than at least one of the reference rectangles S1 to S3), and the cross-sectional shape of the first space 85a can be of various embodiments.

For example, one of the first portion 86a and the second portion 86b may have a curved shape and the other may have a straight shape. The overall shape of the first portion 86a is the curved shape in the embodiment described above, but at least a part of the first portion 86a may have a curved shape, such as a combination of straight and curved shapes. The same applies to the second portion 86b. Although the first portion 86a and the second portion 86b each curve so that they each have the convex shape to the down direction, at least one of them may curve so that it is a convex shape to the up direction. Although the first portion 86a and the second portion 86b each have a convex shape on the same side in the up-down direction, the present invention is not limited to this embodiment, and for example, a shape in FIG. 9 or FIG. 10 may be adopted. In FIG. 9, the first portion 86a and the second portion 86b each have a convex shape toward a side where they approach each other in the up-down direction. In FIG. 9, the second structural portion 88b is thin at both left and right ends in the cross-section perpendicular to the front-rear direction and becomes thicker toward a center in the left-right direction. With this configuration, a upper surface of the second structural portion 88b curves so that the upper surface has a convex shape to the up direction, thereby constituting the second portion 86b of the first space portion 85a. In FIG. 10, the first portion 86a and the second portion 86b each have a convex shape toward a side where they are separated from each other in the up-down direction. In FIG. 10, a lower surface of a first structural portion 88a and a lower surface of a fourth structural portion 88d as a whole are curved so that the lower surfaces have a convex shape to the up direction, thereby constituting the first portion 86a.

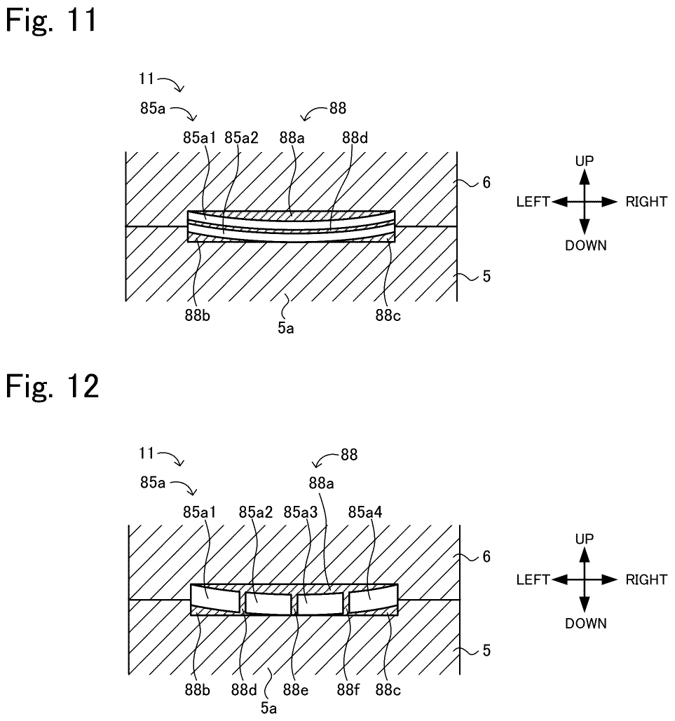

In addition, the first space portion 85a described above may be divided into multiple space portions. For example, as illustrated in FIG. 11, the first space portion 85a may be divided into an upper space portion 85a1 and a lower space portion 85a2 by a fourth structural portion 88d. The fourth structural portion 88d extends over the entire front-rear direction of the first diffusion control section 11, and its left and right edges are connected to the second solid electrolyte layer 6. The fourth structural portion 88d is curved to have a constant thickness and a convex shape to the down direction. The space portions 85al and 85a2 are curved so that each of a first portion (an upper side) and a second portion (a lower side) thereof has a convex shape to the down direction, in the same way as the first space portion 85a illustrated in FIG. 5. With this configuration, a shape of a cross-section of each of the space portions 85a1 and 85a2 that is perpendicular to the front-rear direction is the predetermined shape as described above. For example, by forming three layers that is two vanishing material layers arranged above and below and a structural material layer therebetween, instead of vanishing material layer 185a in FIG. 7A, the space portions 85a1, 85a2, and the fourth structural portion 88d shown in FIG. 11 can be manufactured. In FIG. 11, the first space portion 85a is divided into two spaces, but it may be divided into three or more spaces.

Alternatively, as illustrated in FIG. 12, the first space portion 85a may be divided into four space portions 85al to 85a4 arranged in the left-right direction by the fourth to sixth structural portions 88d to 88f. The fourth to sixth structural portions 88d to 88f each extend across the entire front-rear direction of the first diffusion control section 11, with their upper ends connected to the first structural portion 88a and their lower ends connected to the second structural portion 88b, the third structural portion 88c, or the spacer layer 5. The space portions 85al to 85a4 are curved so that each of a first portion (an upper side) and a second portion (a lower side) thereof has a convex shape to the down direction. With this configuration, a shape of a cross-section of each of the space portions 85al to 85a4 that is perpendicular to the front-rear direction is the predetermined shape as described above. For example, by forming four vanishing material layers and three structural material layers alternately arranged in the left-right direction instead of the vanishing material layer 185a in FIG. 7A, the space portions 85a1 to 85a4 and the fourth to sixth structural portions 88d to 88f in FIG. 12 can be manufactured. In FIG. 12, the first space portion 85a is divided into four spaces, but it may be divided into two or three spaces, or into five or more spaces.

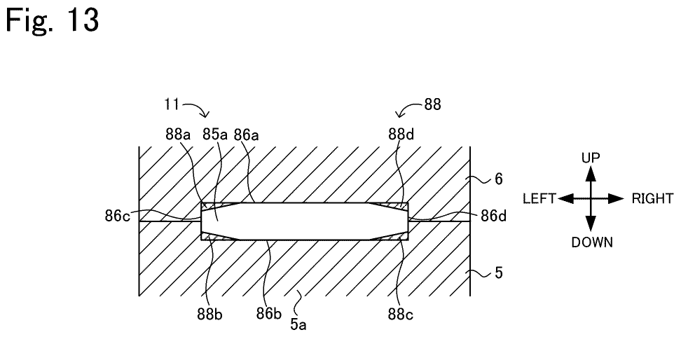

In the embodiment described above, at least a part of the first portion 86a and/or at least a part of the second portion 86b may each have a polygonal line shape rather than a curved shape. For example, an embodiment shown in FIG. 13 may be adopted. In FIG. 13, the first portion 86a of the first space portion 85a is formed with a polygonal line shape having three straight lines and two bending portions, and has a convex shape to the up direction. The first portion 86a is composed of lower surfaces of the first structural portion 88a, the fourth structural portion 88d, and the second solid electrolyte layer 6. The second portion 86b is formed with a polygonal line shape having three straight lines and two bending portions, and has a convex shape to the down direction. The shape of the cross-section perpendicular to the front-rear direction of the first space portion 85a is formed into a polygonal shape (here, an octagonal shape) having five or more sides. The second portion 86b is composed of upper surfaces of the second structural portion 88b, the third structural portion 88c, and the spacer layer 5. In the first space portion 85a of FIG. 13, the first portion 86a and the second portion 86b of the cross-section perpendicular to the front-rear direction each have the polygonal line shape. With this configuration, the shape of this cross-section is the predetermined shape described above (the shape with a longer outer circumference than at least one of the reference rectangles S1 to S3).

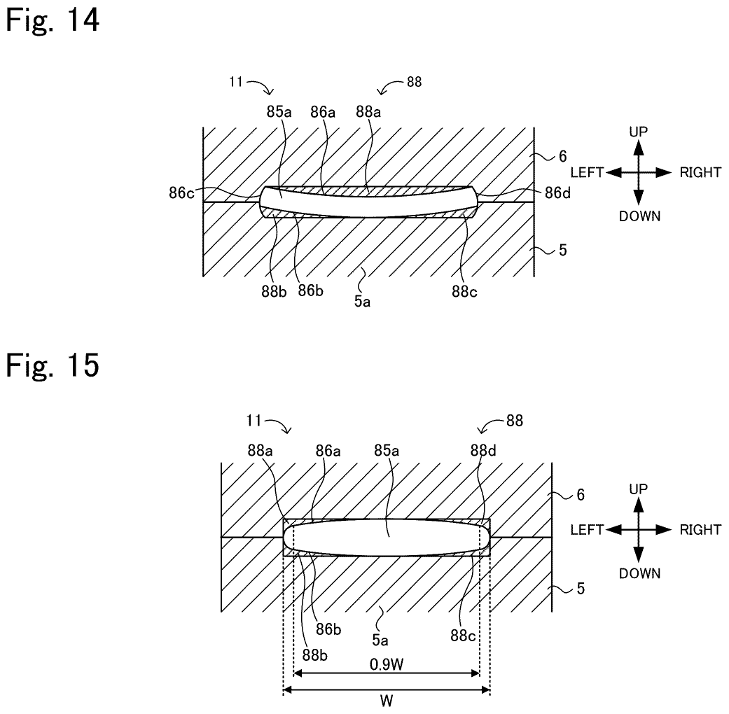

In the embodiment described above, the third portion 86c and the fourth portion 86d each have the straight line shape along the up-down direction in the cross-section perpendicular to the front-rear direction, but the present invention is not limited to this embodiment. For example, a third portion 86c and a fourth portion 86d may each have a curved shape as illustrated in FIG. 14. In FIG. 14, the third portion 86c and the fourth portion 86d each have a convex shape toward a side where they are separated from each other in the left-right direction. For example, by adjusting the viscosity of the green sheets 105 and 106, the vanishing material layer 185a, and the first to third structural material layers 188a to 188c shown in FIG. 7A, the vanishing material layer 185a and the first to third structural layers 188a to 188c can be deformed so as to bulge to the left and right during the pressure bonding process, and the first space portion 85a having the shapes of the third portion 86c and the fourth portion 86d illustrated in FIG. 14 can be formed.

In the embodiment described above, as illustrated in FIG. 5, the boundaries between the first to fourth portions 86a to 86d are corners, and the boundaries are clearly defined, but the present invention is not limited to the embodiment. For example, when the third portion 86c and the fourth portion 86b in FIG. 10 each have the curved shape as shown in FIG. 14, an outer circumference of a cross-section of a first space portion 85a becomes close to an elliptical shape as shown in FIG. 15, and the boundaries between the first to fourth portions 86a to 86d may not be clearly defined. In such a case, as illustrated in FIG. 15, an outer circumference portion existing in the area of 90% of the length (0.9W) that positioning at a central portion in the left-right direction, with a width W of the first space portion 85a in the left-right direction as a reference, are defined as the first portion 86a and the second portion 86b. For example, even if the boundaries between the first portion 86a and the second portion 86b and the remaining portion of the outer circumference of the first space portion 85a is not clear as shown in FIG. 15, when the first portion 86a and the second portion 86b defined in this manner each have a curved shape, the first space portion 85a is considered to have a first portion 86a and a second portion 86b that each have a curved shape.

In the embodiment described above, the first space portion 85a has the first to fourth portions 86a to 86d, but the present invention is not limited to this embodiment. For example, in FIG. 10, the first space portion 85a does not have the third portion 86c and the fourth portion 86d, and the shape of the outer circumference of the cross-section of the first space portion 85a may be a shape in which the first portion 86a and the second portion 86b are directly connected at the left and right ends.

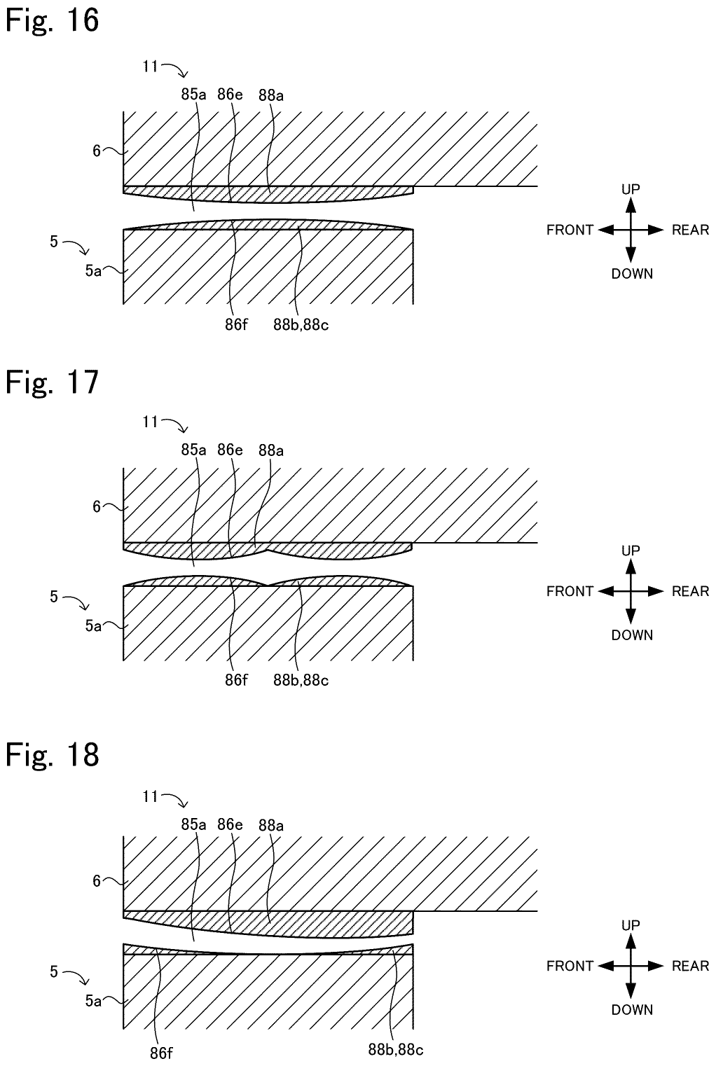

In the embodiment described above, the first space portion 85a has the same shape (the shape shown in FIG. 5) in the cross-section perpendicular to the front-rear direction at any portion in the front-rear direction, but the first space portion 85a is not limited to this embodiment, and it is sufficient if the shape of the cross-section in at least a part of the first space portion 85a in the front-rear direction has the predetermined shape described above. For example, only a front end (upstream end) portion of the first space portion 85a may have the cross-sectional shape illustrated in FIG. 5, and the other portions may have shapes of a rectangular cross-section perpendicular to the front-rear direction. In addition, the first space portion 85a may be formed such that a cross-sectional area of the cross-section perpendicular to the front-rear direction is smaller in at least a part of a downstream side of the upstream end than in the upstream end. For example, a configuration shown in FIG. 16 may be adopted. FIG. 16 shows a cross-section of the first space portion 85a parallel to the front-rear direction. In FIG. 16, the upper and lower outlines 86e and 86f of the cross-section of the first space portion 85a parallel to the front-rear direction each have a curved shape, and the outlines 86e and 86f both extend so as to curve with respect to the front-rear direction. In addition, the outlines 86e and 86f each have a convex shape toward a side where they approach each other in the up-down direction. As a result, the cross-sectional area of the cross-section of the first space portion 85a perpendicular to the front-rear direction is the smallest at a center in the front-rear direction. In this way, the pressure loss of the measurement-object gas passing through a portion of the first space portion 85a having a smaller cross-sectional area than the upstream end (for example, a central portion in the front-rear direction) increases. Therefore, by this shape of this first space portion 85a, the effect of the dynamic pressure of measurement-object gas on the main pump cell 21, auxiliary pump cell 50, and measurement pump cell 41 that are located downstream of the first diffusion control section 11 can be also reduced. The shapes of the outlines 86e and 86f of the first space portion 85a in FIG. 16 can be adjusted by the shapes of the first to third structural portions 88a to 88c, similarly to the shapes of the first portion 86a and the second portion 86b. The first space portion 85a illustrated in FIG. 16 may have the predetermined shape in the cross-section perpendicular to the front-rear direction at any portion in the front-rear direction. For example, the first space portion 85a may have a shape similar to the shape shown in FIG. 5 in the cross-section perpendicular to the front-rear direction at any portion in the front-rear direction.

Instead of the embodiment in FIG. 16, embodiments in FIG. 17 or 18 may be adopted. In FIG. 17, an outline 86e has a curved shape formed by connecting two outlines 86e of FIG. 16 front to back, and an outline 86f has a curved shape formed by connecting two outlines 86f of FIG. 16 front to back. Therefore, the outlines 86e and 86f in FIG. 17 each have two sets of convex shapes toward a side where they approach each other in the up-down direction. As a result, in FIG. 17, there are two locations where the cross-sectional area of the cross-section of the first space portion 85a perpendicular to the front-rear direction is the smallest, and these two locations are located in the middle of the front-rear direction (at positions other than the front end and rear end), and the pressure loss of the measurement-object gas passing through these two locations increases. In FIG. 18, the upper and lower outlines 86e and 86f of the cross-section of the first space portion 85a parallel to the front-rear direction each have a curved shape, and the outlines 86e and 86f both extend so as to curve with respect to the front-rear direction. In addition, the outlines 86e and 86f each have a convex shape on the same side (lower side) in the up-down direction. Furthermore, the outlines 86e and 86f are shaped such that the distance between them in the up-down direction becomes smaller as they approach the rear end relative to the front end. As a result, the first space portion 85a in FIG. 18 has a cross-sectional area of the cross-section perpendicular to the front-rear direction that is the smallest at the rear end. In this way, in FIG. 18, the pressure loss of the measurement-object gas passing through the rear end of the first space portion 85a increases. Each of the first space portions 85a illustrated in FIGS. 16 and 17 may have the predetermined shape in the cross-section perpendicular to the front-rear direction at any portion in the front-rear direction. For example, each of the first space portions 85a may have a shape similar to the shape shown in FIG. 5 in the cross-section perpendicular to the front-rear direction at any portion in the front-rear direction.

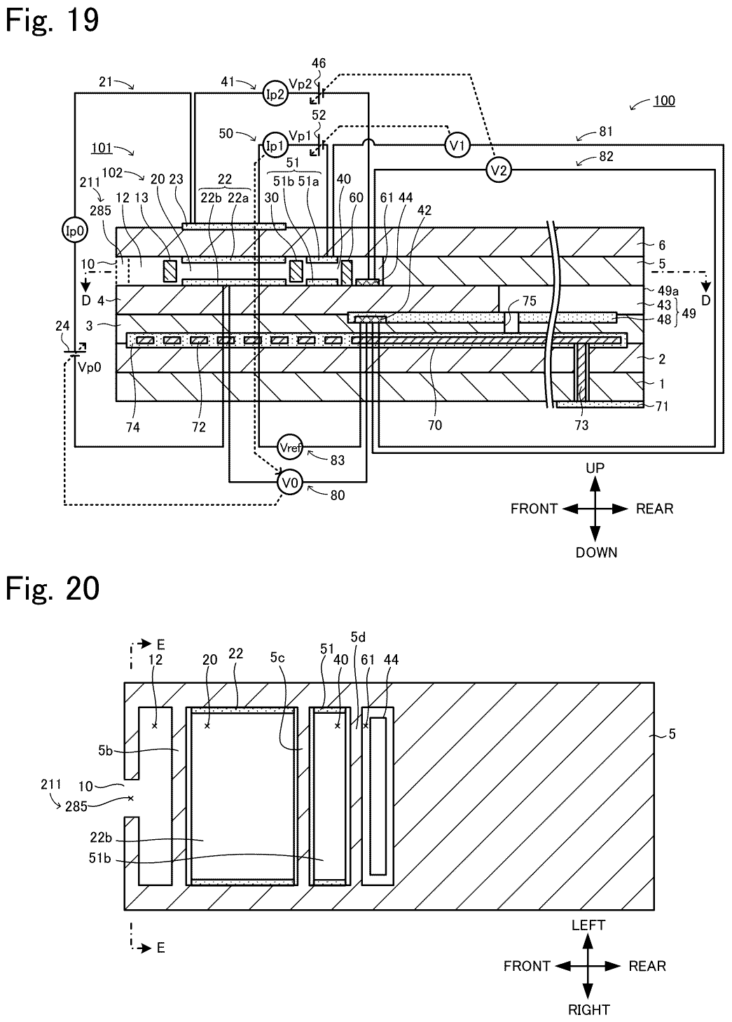

In the embodiment described above, the first diffusion control section 11 has the first space portion 85a provided between the spacer layer 5 and the second solid electrolyte layer 6 and the second space portion 85b provided between the spacer layer 5 and the second solid electrolyte layer 6, but the first diffusion control section 11 is not limited to this embodiment, and the first diffusion control section 11 may has only one of the first space portion 85a and the second space portion 85b. The first space portion 85a of the first diffusion control section 11 is the space provided between the spacer layer 5 and the second solid electrolyte layer 6 that are formed adjacent to each other, but the first space portion 85a is not limited to this embodiment. For example, the first space portion 85a may be a space formed by punching out any of the layers constituting the element body 102. For example, the sensor element 101 may include a first diffusion control section 211 having a space portion 285 illustrated in FIGS. 19 to 21. As illustrated in FIGS. 19 to 21, the space portion 285 of the first diffusion control section 211 is a space formed by punching out the spacer layer 5. Since the space portion 285 has a longer length in the up-down direction than the first space portion 85a, the length in the left-right direction is made shorter than that of the first space portion 85a so that an area of a cross-section of the space portion 285 perpendicular to the front-rear direction approximately becomes the same as that of the first space portion 85a. In addition, the left-right length of the space portion 285 is shorter than the left-right length of the buffer space 12 (see FIG. 20).

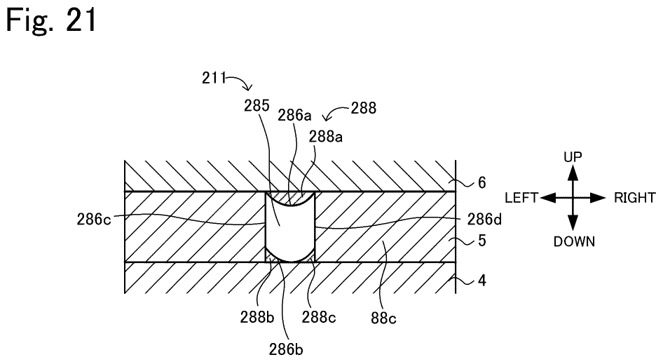

As illustrated in FIG. 21, in the cross-section perpendicular to the front-rear direction, the space portion 285 has first to fourth portions 286a to 286d as an outer circumference of the cross-section. The first portion 286a, the second portion 286b, the third portion 286c, and the fourth portion 286d correspond to the upper, lower, left, and right sides of the outer circumference of the cross-section of the space portion 285. The first portion 286a and the second portion 286b face each other in the up-down direction (the layered direction) and extend along the left-right direction (the second direction), respectively. The third portion 286c and the fourth portion 286d face each other in the left-right direction and extend along the up-down direction, respectively. The first part 286a and the second part 286b are shaped similarly to the first part 86a and the second part 86b in FIG. 5. Specifically, the first part 286a and the second part 286b each have a curved shape and are each curved to form a convex shape on the same side in the up-down direction (in this case, lower side). The third portion 286c and the fourth portion 286d each have a straight line shape along the up-down direction, similarly to the shapes of the third portion 86c and the fourth portion 86d in FIG. 5. The cross-section of the space portion 285 has a length in the up-down direction that is greater than a length in the left-right direction. The shapes of the first portion 286a and the second portion 286b of the space portion 285 can be adjusted by the shapes of a first to third structural portions 288a to 288c that a structural portion 288 includes, similarly to the shapes of the first portion 86a and the second portion 86b. Specifically, the first structural portion 288a is provided on the lower surface of the second solid electrolyte layer 6. The first structural portion 288a is thin at both left and right ends in the cross-section perpendicular to the front-rear direction and becomes thicker toward a center in the left-right direction. With this configuration, a lower surface of the first structural portion 288a thus curves so that the lower surface has a convex shape to the down direction, thereby constituting the first portion 286a of the space portion 285. The second structural portion 288b and the third structural portion 288c are provided on the upper surface of the first solid electrolyte layer 4. The second structural portion 288b is thick at left end in the cross-section perpendicular to the front-rear direction and becomes thinner toward a center of the space portion 285 in the left-right direction. The third structural portion 88c is thick at right end in the cross-section perpendicular to the front-rear direction and becomes thinner toward a center of the space portion 285 in the left-right direction. With these configurations, an upper surface of the second structural portion 288b and an upper surface of the third structural portion 288c as a whole are curved so that the upper surfaces have a convex shape to the down direction, thereby constituting the second portion 286b. In FIG. 19, the structural portion 288 is omitted from the illustration. The third portion 286c and the fourth portion 286d that each have the straight line shape are constituted by not structural portion 288, but are formed as inner peripheral faces (side surfaces) of punch holes provided in the spacer layer 5 (holes penetrating the spacer layer 5 in the up-down direction). As in the first diffusion control section 11 of the above embodiment, in the space portion 285 of this first diffusion control section 211, the first portion 286a and the second portion 286b of the cross-section perpendicular to the front-rear direction each have the curved shape. With this configuration, the shape of this cross-section is the predetermined shape described above (the shape with a longer outer circumference than at least one of the reference rectangles S1 to S3). As a result, a hydraulic diameter of the space portion 285 becomes smaller than a hydraulic diameter of at least one of the reference rectangles S1 to S3, and thus the pressure loss of the measurement-object gas passing through the space portion 285 becomes higher. Therefore, the same effect as that of the first space portion 85a of the above embodiment can be obtained. The shape of the space portion 285 may adopt any of the various embodiments of the first space portion 85a described above.

In the embodiment described above, the cross-sectional shapes of the first space portion 85a and the second space portion 85b of the first diffusion control section 11 are set to the predetermined shape, but the present invention is not limited to this, and the cross-sectional shapes of one or more of the first diffusion control section 11, the second diffusion control section 13, the third diffusion control section 30, and the fourth diffusion control section 60 may be set to the predetermined shape.

In the embodiment described above and FIG. 19, each of the front ends of the first diffusion control sections 11, 211 is a gas inlet 10, but the present invention is not limited to this. For example, the first bridging portion 5a and the first diffusion control section 11 may be disposed behind the gas inlet 10 in the measurement-object gas flow section.

In the embodiment described above, the first space portion 85a, the second space portion 85b, and the space portion 285 all allow the measurement-object gas to flow in the front-rear direction, but the present invention is not limited to this. For example, the gas inlet 10 may be opened on the left or right surface of the element body 102, and the first space portion 85a may allow the measurement-object gas to flow in the left-right direction. In this case, the left-right direction corresponds to the first direction, and the front-rear direction corresponds to the second direction.

In the embodiment described above, the outer pump electrode 23 plays a role as the electrode (also referred to as an outer main pump electrode) to be paired with the inner pump electrode 22 in the main pump cell 21, plays a role as the electrode (also referred to as an outer auxiliary pump electrode) to be paired with the auxiliary pump electrode 51 in the auxiliary pump cell 50, and plays a role as the electrode (also referred to as an outer measurement electrode) to be paired with the measurement electrode 44 in the measurement pump cell 41; however, the present invention is not limited thereto. One or more of the outer main pump electrode, the outer auxiliary pump electrode, and the outer measurement electrode may be provided separately from the outer pump electrode 23 on outer surface of the element body 102 so as to be in contact with the measurement-object gas.

In the embodiment described above, the outer pump electrode 23 is exposed outside the sensor element 101, but the present invention is not limited to this, it is sufficient if the outer pump electrode 23 is provided on the outer surface of the element body 102 so as to be in contact with the measurement-object gas. For example, the sensor element 101 may include a porous protective layer that covers the element body 102 and allows the measurement-object gas to pass through, and the outer pump electrode 23 may also be covered by the porous protective layer.

While the sensor element 101 according to the above embodiment is configured to detect the concentration of NOx in the measurement-object gas, such an embodiment is not limited as long as the sensor element is configured to detect the concentration of any specific gas in the measurement-object gas. For example, the concentration of any oxide other than NOx may be defined as the specific gas concentration. If the specific gas is an oxide, oxygen is produced when the specific gas itself is reduced in the third internal cavity 61, as with the case of the above embodiment. Therefore, the measurement pump cell 41 can detect the specific gas concentration by acquiring a value (for example, the pump current Ip2) detected in correspondence with the oxygen. Alternatively, the specific gas may be a non-oxide, such as ammonia. If the specific gas is a non-oxide, the specific gas is converted into an oxide (e.g., into NO in the case of ammonia), so that oxygen is produced when the converted gas is reduced in the third internal cavity 61. Thus, the measurement pump cell 41 can acquire a value (e.g., the pump current Ip2) detected in correspondence with this oxygen and thus detect the specific gas concentration. For example, since the inner pump electrode 22 in the first internal cavity 20 functions as a catalyst, the ammonia is converted into NO in the first internal cavity 20.

In the above-described embodiment, the element body 102 of the sensor element 101 is a layered body having a plurality of solid electrolyte layers (layers 1 to 6), but is not limited thereto. The element body 102 of the sensor element 101 may include at least one oxygen-ion-conductive solid electrolyte layer. For example, in FIG. 1, the layers 1 to 5 other than the second solid electrolyte layer 6 may be layers (e.g., layers composed of alumina) composed of a material other than solid electrolyte. In this case, the electrodes of the sensor element 101 may be disposed in the second solid electrolyte layer 6. For example, the measurement electrode 44 in FIG. 1 may be disposed on the lower surface of the second solid electrolyte layer 6. Also, the reference gas inlet space 43 may be provided in the spacer layer 5 instead of the first solid electrolyte layer 4, the reference gas inlet layer 48 may be provided between the second solid electrolyte layer 6 and the spacer layer 5 instead of between the first solid electrolyte layer 4 and the third substrate layer 3, and the reference electrode 42 may be provided rearward of the third internal cavity 61 and on the lower surface of the second solid electrolyte layer 6.

The present application claims priority from Japanese Patent Application No. 2024-095786 filed Jun. 13, 2024, the entire contents of which are incorporated herein by reference.

What is claimed is:

1. A sensor element for detecting a concentration of a specific gas in a measurement-object gas, the sensor element comprising:
an element body having a longitudinal direction, being a layered body that is composed of multiple layers stacked in a layered direction perpendicular to the longitudinal direction, the multiple layers including an oxygen-ion-conductive solid electrolyte layer, and having a measurement-object gas flow section inside that introduces the measurement-object gas from a gas inlet and causes the measurement-object gas to flow in a first direction;
a pump cell including an inner electrode provided in an internal cavity of the measurement-object gas flow section and an outer electrode provided on an outer surface of the element body, and
a diffusion control section that is located upstream of the internal cavity in the measurement-object gas flow section and that provides the measurement-object gas introduced from the gas inlet with diffusion resistance,
wherein the diffusion control section includes a space portion through which the measurement-object gas is caused to flow in the first direction,
a shape of a cross-section perpendicular to the first direction in at least a part of the space portion in the first direction is a predetermined shape with a longer outer circumference than a reference rectangle, and
the reference rectangle is a rectangle that has the same area as the cross-section of the space portion, and the reference rectangle is a rectangle that is formed by deforming the circumscribed rectangle of the cross-section, which is composed of two sides parallel to the layered direction and two sides parallel to a second direction that is perpendicular to the layered direction, so that short sides become shorter while maintaining the length of long sides, or a rectangle that is formed by deforming the circumscribed rectangle while maintaining a ratio of each side, or a rectangle that is formed by changing the length of each side of the circumscribed rectangle so as to maximize the overlapping area with the cross-section.

2. The sensor element according to claim 1,
wherein the outer circumference of the predetermined shape includes a first portion and a second portion that face each other in the layered direction and extend along the second direction, respectively, and
at least a part of the first portion and at least a part of the second portion each have a curved shape.

3. The sensor element according to claim 1,
wherein the outer circumference of the predetermined shape includes a first portion and a second portion that face each other in the layered direction and extend along the second direction, respectively, and
at least a part of the first portion and/or at least a part of the second portion each have a polygonal line shape.

4. The sensor element according to claim 2,
wherein the first portion and the second portion each have a convex shape on the same side in the layered direction.

5. The sensor element according to claim 2,
wherein the first portion and the second portion each have a convex shape toward a side where they approach each other or toward a side where they are separated from each other in the layered direction.

6. The sensor element according to claim 1,
wherein the cross-section of the space portion has a length in the second direction that is greater than a length in the layered direction.

7. The sensor element according to claim 1,
wherein the diffusion control section has a plurality of the space portions, and
each of the plurality of the space sections has a shape of the cross-section that is the predetermined shape.

8. The sensor element according to claim 1,
wherein the space portion is formed such that a cross-sectional area of a cross-section perpendicular to the first direction is smaller in at least a part of a downstream side of a upstream end than in the upstream end.

9. The sensor element according to claim 8,
wherein the space portion has an outline of cross-section parallel to the first direction, and the outline extends so as to curve with respect to the first direction.

10. A gas sensor comprising the sensor element according to claim 1.
